(19) United States
(12) Patent Application Publication
(10) Pub. No.: US 2018/0147330 A1
(43) Pub. Date: May 31, 2018

(54) MULTIFUNCTIONAL NEGATIVE PRESSURE DRAINAGE SYSTEM

(71) Applicant: GUANGZHOU SUN-SHING BIOTECH CO., LTD., Guangzhou, Guangdong (CN)

(72) Inventors: YUN PENG, Guangzhou, Guangdong (CN); XI FENG YANG, Guangzhou, Guangdong (CN); CHEN GUANG ZENG, Guangzhou, Guangdong (CN)

(73) Assignee: Guangzhoa Sun-Shing Biotech Co., Ltd., Guangzhou, Guangdong (CN)

(21) Appl. No.: 15/568,296

(22) PCT Filed: Jul. 30, 2015
(86) PCT No.: PCT/CN2015/085535

§ 371 Date: Oct. 20, 2017

(30) Foreign Application Priority Data
Apr. 23, 2015 (CN) 201510202872.0

(51) Int. Cl. A61M 1/00; A61F 13/02; A61M 3/02

(52) U.S. Cl. CPC A61M 1/0084; A61F 13/0216; A61M 1/0066; A61M 1/0088; A61M 3/0279; A61M 2205/15; A61M 2205/331; A61M 2205/3344; A61M 2205/3379; A61M 2205/7518; A61M 2205/7545; A61M 1/0035

(57) ABSTRACT

The present invention discloses a multifunctional negative pressure drainage system switchable between various modes, including continuous negative pressure, intermittent negative pressure, rinsing, drug administration, and drug administration loop, including a medical wound dressing, a waste fluid bottle, a vacuum pump, a draining tube A, an airway A, a draining tube B, a peristaltic pump, a draining tube C, an infusion bottle, a draining tube E, a control valve A, a control valve B, and a control valve F. The medical wound dressing, the draining tube B, the peristaltic pump, the draining tube C, and the infusion bottle are connected in turn. The medical wound dressing, the draining tube A, the waste fluid bottle, the airway A, and the vacuum pump are connected in turn. The draining tube E is connected between the draining tube C and the draining tube A. The control valve A is disposed on the draining tube A. The junction between the draining tube E and the draining tube A is located between the control valve A and the medical wound dressing. The control valve B is disposed on the draining tube C. The junction of the draining tube E and the draining tube C is located between the control valve B and the peristaltic pump. The control valve F is disposed on the draining tube E.

### BACKGROUND OF THE INVENTION

### 1. Field of the Invention

The present invention relates to negative pressure drainage systems, and more particularly, to a multifunctional negative pressure drainage system, which is able to rinse and drain out the wound of the patient, and efficiently switchable between two modes.

### 2. Description of the Related Art

Recently, with the fast paced social and economic developments and the accelerated society aging, more and more acute and chronic skin diseases wound that are difficult to be healed occur, such as the wound caused by diabetic foot ulcers, deep pressure ulcer, or injury. Also, wounds needed to be drained often occur after various surgical operations. Blood, pus, tissue fluid, and excretion possibly accumulate at the lesion or wound. If the fluid are not immediately removed, the healing of the wound will be affected, and further degeneration of the wound surface might occur in a worse case. Presently effectively solution applies a negative pressure draining technique to discharge the fluid. During the surgical operation, a negative pressure draining tube is inserted into the wound location, so as to drain out the accumulate fluid through the negative pressure draining tube.

Generally, a negative pressure drainage apparatus provides only two treatment modes, continuous negative pressure and intermittent negative pressure, wherein the two treatment modes are unable to be programmed to be combined. During the wound treatment, the doctor usually faces various complicated cases of wound damage. For further improving the wound healing and achieving the treatment result, doctor needs to carry out various treating methods, such as medical treatment, inflammation allaying and rinsing treatment, negative draining treatment, debriding treatment, and growth factor treatment, and achieves the most optimized treatment project through an organic combination. The negative drainage treatment apparatuses in the present market fail to achieve such requirement.

### SUMMARY OF THE INVENTION

For improving the aforementioned disadvantages, the present invention provides a multifunctional negative drainage system which is efficiently switchable between various modes, including continuous negative pressure, intermittent negative pressure treatment, rinsing treatment, drug administration treatment, and drug administration loop treatment, for treating the wound of a patient; also, a combination of two or more treatment modes is selectable for carrying out a programmable treatment. The present invention includes a medical wound dressing, a waste fluid bottle, a vacuum pump, a draining tube A, and an airway A. The medical wound dressing is adhered to a wound location of a patient. The draining tube A is connected between the medical wound dressing and the waste fluid bottle. The airway A is connected between the waste fluid bottle and the vacuum pump. An infusion bottle, a draining tube C, a control valve B, and a control valve A are further included. The draining tube C is connected between the infusion bottle and the medical wound dressing. The control valve B is disposed on the draining tube C. The control valve A is disposed on the draining tube A.

To further achieve the present invention, the infusion bottle is further provided with a liquid level sensor B for monitoring the liquid level in the infusion bottle.

To further achieve the present invention, a draining tube B and a peristaltic pump are further included. The medical wound dressing, the draining tube B, the peristaltic pump, the draining tube C, and the infusion bottle are connected in turn.

To further achieve the present invention, a draining tube B, a peristaltic pump, a draining tube E, and a control valve F are further included. The draining tube B is connected between the peristaltic pump and the medical wound dressing. The draining tube C is connected between the peristaltic pump and the infusion bottle. The draining tube E is connected between the draining tube C and the draining tube A. The control valve A is disposed on the draining tube A, and the junction between the draining tube E and the draining tube A is located between the control valve A and the medical wound dressing. The control valve B is disposed on the draining tube C, and the junction between the draining tube E and the draining tube C is located between the control valve B and the peristaltic pump. The control valve F is disposed on the draining tube E.

To further achieve the present invention, the waste fluid bottle is provided with a turbidity sensor for immediately monitoring the turbidity of the waste fluid in the waste fluid bottle.

To further achieve the present invention, the waste fluid bottle is provided with a liquid level sensor A for immediately monitoring the liquid level of the waste fluid in the waste fluid bottle.

To further achieve the present invention, a pressure sensor and an airway B are further included. The airway B has one end thereof connected to the medical wound dressing, with the other end thereof connected to the pressure sensor, so as to immediately monitor the strength of the negative pressure at the wound location of the patient.

To further achieve the present invention, an airway D, a control valve E, and a filter tip are further included. The control valve E is disposed on the airway D. The airway D has one end thereof connected to the medical wound dressing, with the other end thereof connected to the filter tip.

To further achieve the present invention, an airway C and a bacteria screening device are further included. The airway C is connected between the medical wound dressing and the bacteria screening device. The airway D is connected between the bacteria screening device and the filter tip.

The present invention achieves following advantages:

1. The negative pressure drainage system of the present invention, by use of the switch of the control valve A and the control valve B, is efficiently switchable between various modes, including continuous negative pressure, intermittent negative pressure treatment, rinsing treatment, drug administration treatment, and drug administration loop treatment, for treating the wound of a patient. Also, a combination of two or more treatment modes is selectable for carrying out a programmable treatment, achieving the operation simplicity and facilitating the convenience of usage for the medical staff and the medical equipment operation.

2. The negative pressure drainage system of the present invention, by use of the switch of the control valve A, the control valve B, and the control valve F, is freely switchable among the single infusion mode, the continuous negative pressure mode, and the self-looping mode. Under the self-looping mode, the peristaltic pump is applied for facilitating the waste fluid flowing loop at the wound location, so as to continuously stimulate the wound location to generate a continuous fluid shear force, thereby accelerating the nutrient (medicine) absorption of the wound to enhance the healing of the wound.

3. The negative pressure drainage system of the present invention includes a turbidity sensor, so as to immediately monitor the turbidity of the waste fluid in the waste fluid bottle with the turbidity sensor, allowing medical staff or medical equipment to identify the recovering status of the wound of the patient according to the data sensed by the turbidity sensor.

4. The negative pressure drainage system of the present invention includes the liquid level sensor A disposed in the waste fluid bottle. When the waste liquid in the waste liquid bottle is about to be full, the liquid level sensor A is triggered to remind the medical staff or the remote host machine to replace the waste fluid bottle, thus conforming to the development of the fully automatic intelligent medical equipment in the future.

5. The negative pressure drainage system of the present invention includes a pressure monitoring system, so as to immediately monitor the actual strength of the negative pressure at the wound location with the pressure monitoring system and send the actual strength of the pressure to the host machine, allowing the host machine to efficiently control the strength of the negative pressure of the wound location, thereby preventing the excessive pressure at the wound from damaging the tissue of the patient, and also effectively monitoring if any block or air leakage of the drainage system occurs. When the data sensed by the pressure sensor fails to reach a predetermined negative pressure value in a long time, and if the negative pressure value is unable to remain consistent when the host machine is temporarily halted, the system is considered to be air leaking. If the negative pressure remains consistent by fails to reach the predetermined negative pressure value, the system is considered to be blocked, such that the negative pressure tube shall be replaced or rinsed.

6. The negative pressure drainage system of the present invention, by use of the switch of the control valve, is freely switchable between the continuous negative pressure mode, intermittent negative pressure mode, full-speed rinsing mode, controllable rinsing mode, single infusion mode, and looping mode, so as to provide a proper operation mode selected based on the recovering stage or treatment timing of the patient, achieving an improved general utility and enhancing the recovering of the patient.

7. When the negative pressure drainage system of the present invention is in the continuous negative pressure mode, the target pressure value at the wound location is allowed to be set by use of the host machine. By immediately monitoring the actual strength of the pressure at the wound location by use of the pressure sensor, when the actual pressure strength differs from the target pressure value, the output performance of the vacuum pump is varied through the host machine for adjusting the actual strength of the pressure at the wound location, thereby ensuring the actual strength of the pressure to be consistent with the target pressure value.

8. When the negative pressure drainage system of the present invention is in the intermittent negative pressure mode, by use of an alternate variation of the air pressure, an intermittent negative pressure is formed at the wound location, so as to carry out a regular physical stimulation upon the newborn tissue at the wound location, thereby achieving a physically healing effect.

9. When the negative pressure drainage system of the present invention is in the full-speed rinsing mode, the rinsing and disinfecting operation upon the wound location are achieved by the infusion tunnel formed of the infusion bottle, the draining tube C, and the draining tube D. Therefore, when the present invention sucks waste fluid by use of negative pressure, the wound location also undergoes a rinsing process, accelerating the healing of the wound location, also preventing the necessity to unfold the wound dress at the wound location of the patient and cleanse the wound with other tools, so as to greatly lower the pain of the patient.

10. When the negative pressure drainage system of the present invention is in the controllable rinsing mode, the infusing speed of the infusion bottle is effectively controlled through the peristaltic pump, so as to carry out a proper rinsing operation based on the recovering status of the wound location (different rinsing speeds are needed for different recovering stage), thereby saving cost and lowering wasted.

11. When the negative pressure drainage system of the present invention is in the single infusion mode, the rinsing and disinfecting operation upon the wound location are carried out through the infusion tunnel formed of the infusion bottle, draining tube C, and draining tube B. Also, the output performance of the peristaltic pump is controlled through the host machine, so as to effectively control the infusing speed of the infusion bottle (rinsing speed for the wound location).

### BRIEF DESCRIPTION OF THE DRAWINGS

FIG. 1 is a schematic view illustrating the present invention being in the continuous negative pressure mode.

FIG. 2 is a schematic view illustrating the present invention being in the intermittent negative pressure mode.

FIG. 3 is a schematic view illustrating the present invention being in the full-speed rinsing mode.

FIG. 4 is a schematic view illustrating the present invention being in the controllable rinsing mode.

FIG. 5 is a schematic view illustrating the present invention being in the single infusion mode.

FIG. 6 is a schematic view illustrating the present invention being in the looping mode.

### DETAILED DESCRIPTION OF THE INVENTION

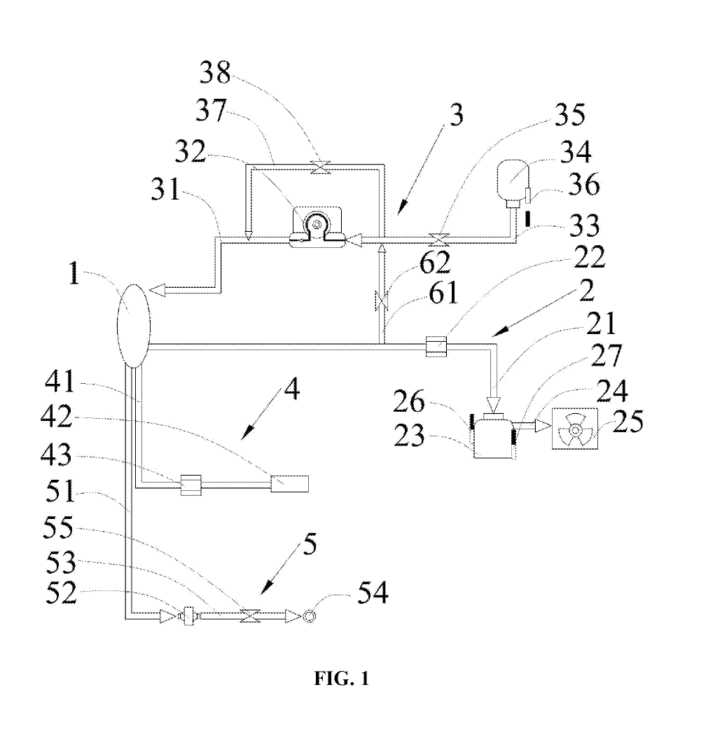

The present invention is explained in conjunction with the accompanying drawings as following, wherein the direction referred in the present invention is defined according to FIG. 1.

Referring to FIG. 1 to FIG. 6, a multifunctional negative pressure drainage system in accordance with the present invention comprises a medical wound dressing 1, a drainage system 2, a rinse system 3, a pressure monitoring system 4, and a pressure balance system 5. The medical wound dressing 1 is adhered to a wound location of a patient. The drainage system 2 is applied for draining out the blood and waste fluid accumulation at the wound location of the patient. The rinse system 3 is applied for disinfecting and rinsing the wound location of the patient. The pressure monitoring system 4 is applied for monitoring the actual strength of pressure at the wound location of the patient. The pressure balance system 5 is applied to balance the strength of pressure at the wound location of the patient, so as to prevent the pressure at the wound location from being excessive.

The medical wound dressing 1 is allowed to be a porous sponge wound dressing or a combination wound dressing of porous sponge and separation pad. The separation pad usually applies Vaseline gauze, or a loose and porous separation pad formed by pierced hydrophilic or liquid absorbing gel. The separation pad is highly bio-compatible and has a hydrophilic, soft, smooth, Nano-porous surface, which is able to be a drug carrier.

The drainage system 2 includes a draining tube A 21, a control valve A 22, a waste fluid bottle 23, an airway A 24, a vacuum pump 25, a liquid level sensor A 26, and a turbidity sensor 27. The draining tube A 21 has one end thereof passing through the medical wound dressing 1 and inserted into the wound location, with the other end of the draining tube A 21 connected with the waste fluid bottle 23, so as to draining the waste fluid at the wound location into the waste fluid bottle 23. The control valve A 22 is disposed on the draining tube A 21 for controlling the waste fluid flowing into the waste fluid bottle 23. The waste fluid bottle 23 is applied for gathering the waste fluid from the wound location for facilitating a periodic cleaning process. The airway A 24 is connected between the waste fluid bottle 23 and the vacuum pump 25. The vacuum pump 25 is applied for forming a negative pressure in the waste fluid bottle 23, so as to sucking the waste fluid from the wound location into the waste fluid bottle 23 by use of the negative pressure. The liquid level sensor A 26 is disposed on the outer wall of the waste fluid bottle 23 for immediately monitoring the gathered amount of the waste fluid in the waste fluid bottle 23. When the waste fluid is about to be full in the waste fluid bottle 23, the liquid level sensor A 26 is triggered and sends a control signal indicating “waste fluid bottle full”, so as to remind the medical staff to replace the waste fluid bottle 23 or to empty the waste fluid in the waste fluid bottle 23. The turbidity sensor 27 is disposed in the waste fluid bottle 23 for immediately monitoring the turbidity of the waste fluid in the waste fluid bottle 23. The turbidity sensor 27 sends the immediately monitored turbidity value to the host machine, such that the recovering status of the wound location is determined based on the turbidity value of the waste fluid monitored through the turbidity sensor 27. When the value monitored by the turbidity sensor 27 is lower (the waste fluid is clearer), the accumulated blood and fluid at the wound location are deemed lesser, indicating an improved wound recovering status. When the value monitored by the turbidity sensor 27 is higher (the waste fluid is more turbid), the accumulated blood and fluid at the wound location remain in a large amount, indicating a less optimized wound recovering status. Therefore, no other tools are needed to monitor the recovering status of the wound location, achieving a humanized design and a more accurate determination result.

The rinse system 3 includes a draining tube B 31, a peristaltic pump 32, a draining tube C 33, an infusion bottle 34, a control valve B 35, a liquid level sensor B 36, a draining tube D 37, and a control valve C 38. The draining tube B 31 has one end thereof passing through the medical wound dressing 1 and inserted into the wound location, with the other end of the draining tube B 31 connected to an output end of the peristaltic pump 32. The peristaltic pump 32 is connected between the draining tube B 31 and the draining tube C 33, so as to control the infusing speed by the peristaltic pump 32. The draining tube C 33 has one end thereof connected to the an input end of the peristaltic pump 32, with the other end of the draining tube C 33 connected to the infusion bottle 34. The infusion bottle 34 is applied for storing cleansing solution or disinfectant. The control valve B 35 is disposed on the draining tube C 33 for controlling the infusing condition and speed of the draining tube C 33. The liquid level sensor B 36 is dispose on the outer wall of the infusion bottle 34 for immediately monitoring the residual amount of the cleansing solution or disinfectant. When the cleansing solution or disinfectant in the infusion bottle 34 is running out, the liquid level sensor B 36 is triggered and sends a control signal of “infusion bottle empty” to the host machine, so as to remind the medical staff to replace the infusion bottle 34 or refill the infusion bottle 34 with cleansing solution or disinfectant. The two ends of the draining tube D 37 are connected with the draining tube B 31 and the draining tube C 33, respectively, so as to automatically carry out the infusing progress when the peristaltic pump 32 is not working. The control valve C 38 is disposed on the draining tube D 37 for controlling the infusing status and speed of the draining tube D 37.

The pressure monitoring system 4 includes an airway B 41, a pressure sensor 42, and a control valve D 43. The airway B 41 has one end thereof disposed on one side of the medical wound dressing 1 facing the wound, with the other end thereof connected with the pressure sensor 42. The pressure sensor 42 is applied for immediately monitoring the actual strength of pressure, and sending the monitored actual strength of the pressure to the host machine, allowing the host machine to effectively control the strength of the negative pressure at the wound location, so as to prevent the tissue of the patient from being damaged due to excessive pressure. Also, possible blockage of the drainage system 2 is effectively monitored. (When the system is stably working in a certain status, a larger fluctuation of the data monitored by the pressure sensor 42 indicates a blockage of the drainage system 2 which needs to be cleared.) The control valve D 43 is disposed on the airway B 41 for controlling the operation status of the pressure sensor 42.

The pressure balance system 5 includes an airway C 51, a bacteria screening device 52, an airway D 53, a filter tip 54, and a control valve E 55. The airway C 51 has one end disposed on one side of the medical wound dressing 1 facing the wound, with the other end thereof connected with the bacteria screening device 52. The bacteria screening device 52 screens the air flowing into the wound to prevent the wound tissue from infection of bacteria. The airway D 53 has one end thereof connected to the bacteria screening device 52, with the other end thereof connected to the filter tip 54. The filter tip 54 is applied for filtering large particles in the air flowing into the wound for preventing the tissue at the wound location from being polluted. The control valve E 55 is disposed on the airway D 53 for controlling the operation status of the airway D 53. (the pressure balance system 5).

When the wound is recovering well, a continuous stimulation is allowed to be carried out upon the wound, so as to generate a continuous fluid shear force upon the wound location to achieve a physically healing enhancement function, thereby accelerating the healing after a surgical operation. The present invention is able to be provided with a looping mode. In other words, a draining tube E 61 is added between the draining tube C 33 and the draining tube A 21, with a control valve F 62 being disposed on the draining tube E 61. The draining tube E 61 has one end thereof located between a section of the draining tube C 33 between the peristaltic pump 32 and the control valve B 35, with the other end thereof located between the medical wound dressing 1 and the control valve A 22.

The peristaltic pump, the vacuum pump, and all the control valves are systematically controlled through the host machine. All the pressure sensors, the liquid level sensors, and the turbidity sensors are electrically connected with the host machine. All the pressure sensors, the liquid sensors, and the turbidity sensors send the monitored signals to the host machine to be identified by the host machine.

Referring to FIG. 1, the present invention is in the continuous negative pressure mode. The control valve A 22 and the control valve D 43 are in an open status. The control valve B 35, the control valve C 38, the control valve E 55, and the control valve F 62 are in an off status. The vacuum pump 25 and the pressure sensor 42 are in an operation status. The peristaltic pump 32 is in a non-operation status. The present invention sets a target pressure value at the wound location through the host machine, and immediately monitors the actual strength of the pressure at the wound location by use of the pressure sensor 42. When the actual pressure strength differs from the target pressure value, the output performance of the vacuum pump 25 is varied through the host machine, so as to adjust the actual pressure strength at the wound location, thereby ensuring that the actual pressure strength conforms to the target pressure value. Therefore, when discharging the waste fluid by the negative pressure, the actual strength of the pressure is more accurately controlled, so as to prevent the tissue of the patient from being damaged by excessive pressure at the wound location, and effectively monitoring if any blockage of the drainage system 2 occurs at the same time. (When the system is stably working in a certain status, a larger fluctuation of the data monitored by the pressure sensor 42 indicates a blockage of the drainage system 2 which needs to be cleared.)

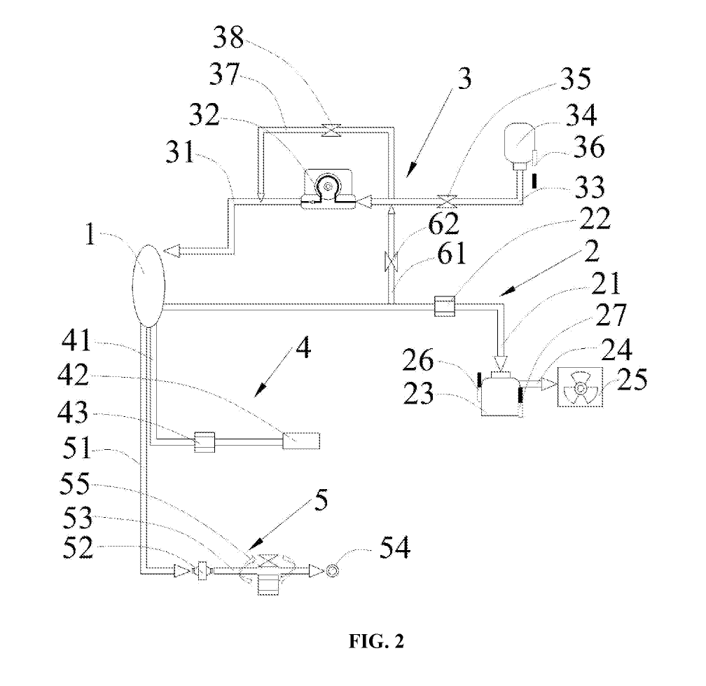

Referring to FIG. 2, the present invention is in the intermittent negative pressure mode. The control valve A 22 and the control valve D 43 are in an open status. The control valve B 35, the control valve C 38, and the control valve F 62 are in an off status. The control valve E 55 is switchable between an on and off status, so that the variation of the control valve E 55 between different operation modes controls the switching between the negative pressure status and the pressure relieving status. The vacuum pump 25 and the pressure sensor 42 are in an operation status. The peristaltic pump 32 is in a non-operation status. In the initial status, the control valve E 55 is in an off status. The target pressure value at the wound location and a time interval value are set through the host machine. The pressure sensor 42 is applied for immediately monitoring the actual strength of the pressure at the wound location. When the actual pressure strength differs from the target pressure value, the output performance of the vacuum pump 25 is varied by use of the host machine to adjust the actual pressure strength at the wound location, thereby ensuring that the actual pressure strength conforms to the target pressure value, wherein the present invention is in the negative pressure status. When the time duration of the negative pressure status reaches the time interval, the control valve E 55 varies the operation status (changes to the open status), and the filter tip 54 is applied to remove the pressure at the wound location, wherein the present invention is in the pressure relieving status. When the time duration of the pressure relieving status reaches the pre-set time interval, The control valve E 55 again varies the operation status (changes to the off status), wherein the present invention is again in the negative pressure status. Repeatedly, the intermittent negative pressure environment is achieved. Therefore, the intermittent negative pressure at the wound location is formed by use of the alternate variation of the air pressure, so as to carry out a regular physical stimulation upon the newborn tissue at the wound location, thereby achieving the physical healing enhancement function.

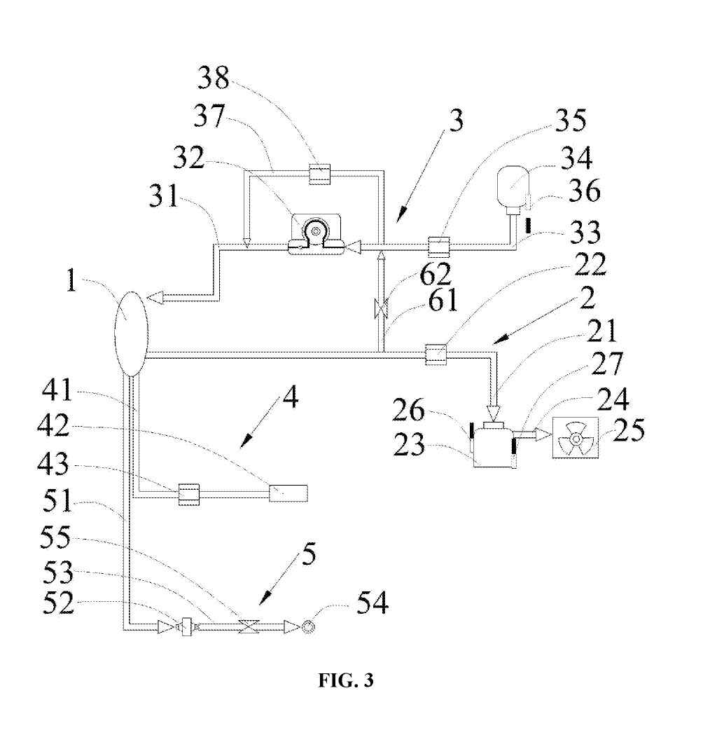

Referring to FIG. 3, the present invention is in the full-speed rinsing mode. The control valve A 22, the control valve B 35, the control valve C 38, and the control valve D 43 are in an open status. The control valve E 55 and the control valve F 62 are in an off status. The vacuum pump 25 and the pressure sensor 42 are in an operation status. The peristaltic pump 32 is in the non-operation status. The present invention is able to carry out the rinsing and disinfecting process through the infusion tunnel formed of the infusion bottle 34, the draining tube C 33, and the draining tube D 37. Further, the target pressure value is set by use of the host machine. The pressure sensor 42 immediately monitors the actual strength of the pressure at the wound location. When the actual pressure strength differs from the target pressure value, the output performance of the vacuum pump 25 is varied by use of the host machine to adjust the actual pressure strength at the wound location, thereby ensuring that the actual pressure strength conforms to the target pressure value. Therefore, when the present invention sucks the waste fluid by the negative pressure, the wound location is rinsed at the same time, so as to facilitate an efficiently recovering of the wound location, thereby preventing the necessity to unfold the wound dress at the wound location of the patient and cleanse the wound with other tools, so as to greatly lower the pain of the patient.

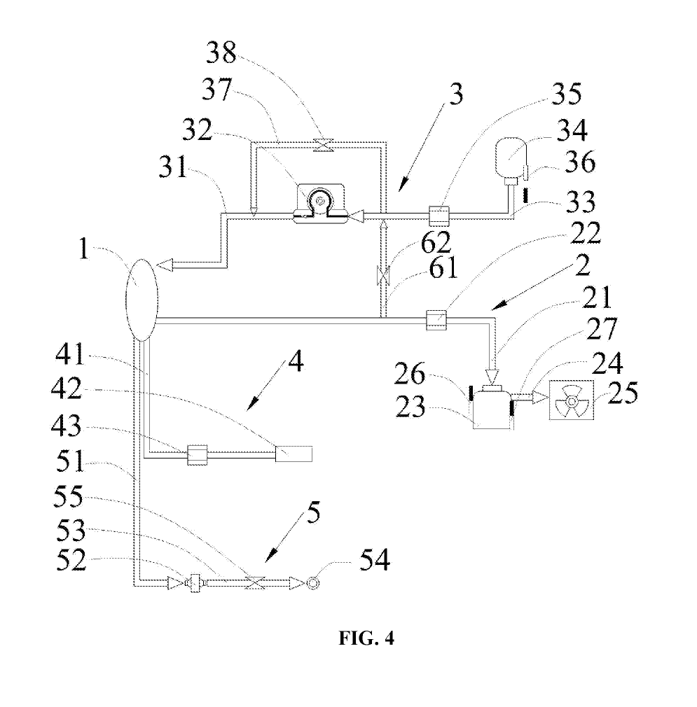

Referring to FIG. 4, the present invention is in the controllable rinsing mode. The control valve A 22, the control valve B 35, and the control valve D 43 are in an opens status. The control valve C 38, the control valve E 55, and the control valve F 62 are in an off status. The vacuum pump 25, the peristaltic pump 32, and the pressure sensor 42 are in an operation status. The present invention controls the output performance of the peristaltic pump 32 by use of the host machine, so as to control the infusing speed of the infusion bottle 34 (rinsing speed at the wound location). Further, the target pressure value is set through the host machine. The pressure sensor 42 is applied to immediately monitor the actual strength of the pressure at the wound location. When the actual pressure strength differs from the target pressure value, the output performance of the vacuum pump 25 is varied by use of the host machine to adjust the actual pressure strength at the wound location, thereby ensuring that the actual pressure strength conforms to the target pressure value. Therefore, when the present invention sucks the waste fluid by the negative pressure, the wound location is rinsed at the same time, so as to facilitate an efficiently recovering of the wound location, thereby preventing the necessity to unfold the wound dress at the wound location of the patient and cleanse the wound with other tools, so as to greatly lower the pain of the patient. Also, by effectively controlling the infusing speed of the infusion bottle 34 through the peristaltic pump 32, a proper rinsing operation according to the recovering status of the wound location is carried out (different rinsing speed is needed in different recovering stage), thus saving the cost and lowering the waste.

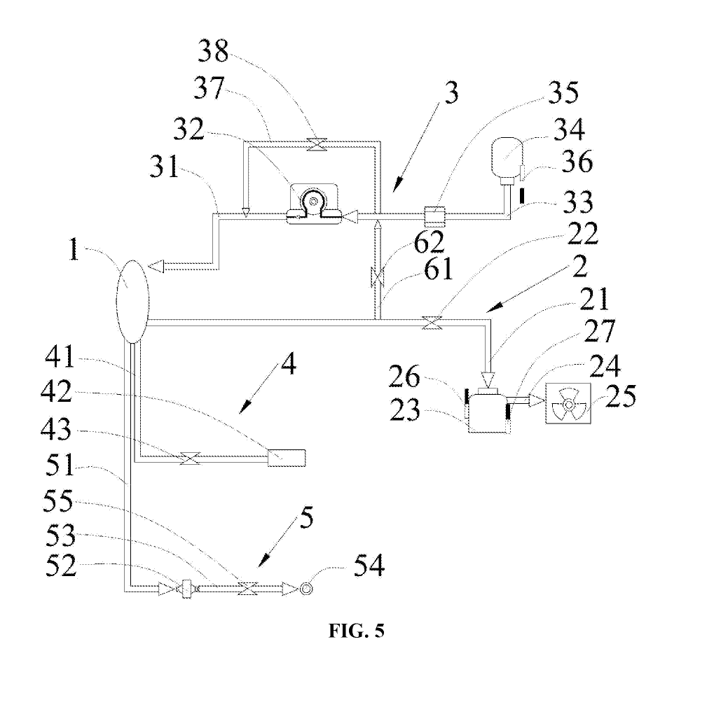

Referring to FIG. 5, the present invention is in the single infusion mode. The control valve B 35 is in an open status. The control valve A 22, the control valve C 38, the control valve D 43, the control valve E 55, and the control valve F 62 are in an off status. The peristaltic pump 32 is in an operation status. The vacuum pump 25 and the pressure sensor 42 are in a non-operation status. Therefore, the present invention is able to carry out the rinsing and disinfecting process upon the wound location through the infusion tunnel formed of the infusion bottle 34, the draining tube C 33, and the draining tube B 31. Also, the output performance of the peristaltic pump 32 is controlled by use of the host machine, so as to effectively control the infusing speed of the infusion bottle 34 (the rinsing speed upon the wound location).

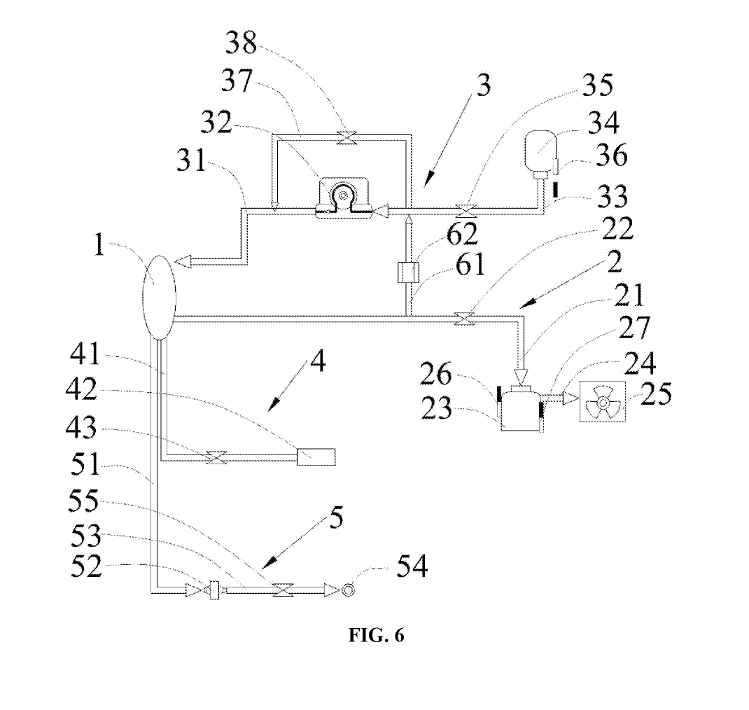

Referring to FIG. 6, the present invention is in the looping mode. The control valve F 62 is in an open status. The control valve A 22, the control valve B 35, the control valve C 38, the control valve D 43, and the control valve E 55 are in an off status. The peristaltic pump 32, the vacuum pump 25, and the pressure sensor 42 are in a non-operation status. Therefore, the waste fluid flowing loop at the wound location is achieved by use of the peristaltic pump 32, so as to continuously stimulate the wound location to generate the continuous fluid shear force at the wound location, thereby improving the absorption of the nutrient (medicine) at the wound location and accelerating the healing of the wound.

Although preferred embodiments of the invention have been described above, the present invention is not limited to the aforementioned embodiments. Various partial or trivial structural modifications and enhancements may be made during the execution. If modifications or variations of the present invention are made without departing from the spirit and scope of the invention and belong to the same technical scope, such modifications and variations are included in the present invention.

What is claimed is:

1. A multifunctional negative pressure drainage system, comprising:
a medical wound dressing, a waste fluid bottle, a vacuum pump, a draining tube A, and an airway A; the medical wound dressing is adhered to a wound location of a patient; the draining tube A is connected between the medical wound dressing and the waste fluid bottle; the airway A is connected between the waste fluid bottle and the vacuum pump,
characterized in that:
further comprising an infusion bottle, a draining tube C, a control valve B, and a control valve A; the draining tube C is connected between the infusion bottle and the medical wound dressing; the control valve B is disposed on the draining tube C; the control valve A is disposed on the draining tube A.

2. The multifunctional negative pressure drainage system of claim 1, characterized in that: the infusion bottle is provided with a liquid level sensor B for monitoring a liquid level in the infusion bottle.

3. The multifunctional negative pressure drainage system of claim 1, characterized in that: further comprising a draining tube B and a peristaltic pump; the medical wound dressing, the draining tube B, the peristaltic pump, the draining tube C, and the infusion bottle are connected in turn.

4. The multifunctional negative pressure drainage system of claim 1, characterized in that: further comprising a draining tube B, a peristaltic pump, a draining tube E, and a control valve F, the draining tube B connected between the peristaltic pump and the medical wound dressing, the draining tube C connected between the peristaltic pump and the infusion bottle, the draining tube E connected between the draining tube C and the draining tube A, the control valve A disposed on the draining tube A, a junction of the draining tube E and the draining tube A located between the control valve A and the medical wound dressing, the control valve B disposed on the draining tube C, a junction of the draining tube E and the draining tube C located between the control valve B and the peristaltic pump, the control valve F disposed on the draining tube E.

5. The multifunctional negative pressure drainage system of claim 1, characterized in that: the waste fluid bottle is provided with a turbidity sensor for immediately monitoring a turbidity of a waste fluid in the waste fluid bottle.

6. The multifunctional negative pressure drainage system of claim 1, characterized in that: the waste fluid bottle is provided with a liquid level sensor A for immediately monitoring a liquid level of a waste fluid in the waste fluid bottle.

7. The multifunctional negative pressure drainage system of claim 1, characterized in that: further comprising a pressure sensor and an airway B, the airway B having one end connected with the medical wound dressing with the other end of the airway B connected with the pressure sensor for monitoring a strength of a negative pressure at the wound location of the patient.

8. The multifunctional negative pressure drainage system of claim 1, characterized in that: further comprising an airway D, a control valve E, and a filter tip; the control valve E is disposed on the airway D; the airway D has one end thereof connected with the medical wound dressing, with the other end of the airway D connected with the filter tip.

9. The multifunctional negative pressure drainage system of claim 8, characterized in that: further comprising an airway C and a bacteria screening device, the airway C connected between the medical wound dressing and the bacteria screening device, the airway D connected between the bacteria screening device and the filter tip.
